(19) United States
(12) Patent Application Publication
(10) Pub. No.: US 2023/0383577 A1
(43) Pub. Date: Nov. 30, 2023

(54) DEVICE AND SYSTEM FOR MONITORING PERSONS IN CUSTODY

(71) Applicant: Richard Dakow, Eastport, NY (US)

(72) Inventor: Richard Dakow, Eastport, NY (US)

(21) Appl. No.: 17/828,624

(22) Filed: May 31, 2022

(51) Int. Cl. E05B 75/00; G16H 40/67; H04W 4/029

(52) U.S. Cl. CPC E05B 75/00; G16H 40/67; H04W 4/029

(57) ABSTRACT

A device and system, attachable to or incorporated into a device used to restrain and monitor and individual which may provide data concerning such restrained individual such as biometric data, location information, device status information and the like. The device of the present invention may be incorporated into and be a part of a standard set of handcuffs, for use in the restraint, management and control of individuals who are in law enforcement custody or control such as detainees, arrestees, prisoners, and the like.

### FIELD OF THE INVENTION

This invention is directed to a device and system, attachable to or incorporated into a standard set of handcuffs, for use in the restraint, management and control of individuals who are in law enforcement custody or control such as detainees, arrestees, prisoners, and the like.

The primary purpose of the device and system is to monitor biometric data concerning the wellbeing of an individual place in restraints, as well as provide location and other ancillary information to those parties responsible for placing the restrained individuals under their custody and or control. in police custody or control such as arrestees, prisoners, and the like.

### BACKGROUND OF THE INVENTION

The need to restrain an individual and the use of a form of restraint such as handcuffs, may arise in varied circumstance such as an arrest, the transfer of a prisoner and the like. In addition, the need to monitor restrained individuals as to the location and/or their biometric condition often arises. Prior art systems for accomplishing the goals of the present invention, while they involve handcuffs of one form or another, do so with devices that are, by design, complicated and unique in their method of manufacture and are not capable of being incorporated into already existing technology. In addition, such prior art technology is limited to sensing the condition of the device itself and not the condition of the restrained.

One such prior art device is found in U.S. Pat. No. 10,458,154 (“Carpino, et al). This patent teaches a restraining device that had the sensing means incorporated within the construction of device and is for the sole purpose of sensing the condition of the device itself.

It is therefore apparent that there is a need for a way for law enforcement to safely monitor restrained individuals as to their status, location, and wellbeing than currently exists in the art.

### SUMMARY OF THE INVENTION

A device and system for monitoring person in custody is provided that includes a sensing element incorporated into a restraining device (such as a pair of hand cuffs), which sensing element can communicate data about the restrained individual such location and biometric data, as well as information concerning the status of the restraining device itself.

One object of the present invention is to provide such a device and system that may be used in combination with known handcuff technology.

A further object of the present invention is to provide such a device and system that is easy to use with known technology.

A still further object of the present invention is to provide such a device and system that may be used with existing “off the shelf” restraining devices without the need to further modify such devices, or alternatively may be integrated into new restraining devices.

### DESCRIPTION OF THE DRAWINGS

These and other features, aspects and advantages of the present invention will become better understood with regard to the following description, appended claims and accompanying drawings where:

FIG. 1 is a front view of a typical pair of law enforcement hand cuffs.

FIG. 2 is a perspective view of one of the cuffs of the hand cuffs shown in FIG. 1.

FIG. 3 is a view of the sensing module employed in the present invention.

FIG. 4 is a top view of the sensing module of FIG. 3.

FIG. 5 is a side view of the sensing module of FIG. 3, taken along line A-A.

### DETAILED DESCRIPTION OF THE INVENTION

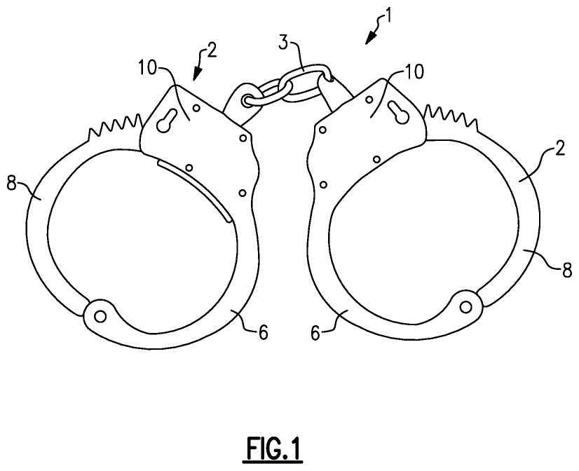

Referring to the drawings, which are for the purpose of illustrating preferred embodiments of the invention only, and not for the purpose of limiting same, FIG. 1-5 show the subject device in its preferred embodiment. Reference numerals, within the figures, are used to identify to the details of this preferred embodiment. However, it will be evident that this does not limit the subject invention to the specific details provided in the FIGURES.

FIG. 1. is an illustration of a typical pair of handcuffs 1, in use by law enforcement, and others today, for restraining individuals. It is comprised two individual, lockable cuffs 2 joined together by a chain 4 or other joining means. Each cuff 2 has a first section 6 and a second section 8 hinged to the first section 6. Each cuff 2 also includes a locking mechanism 10 to lock second section 8 into first section 6 around the wrist or ankles of the restrained individual.

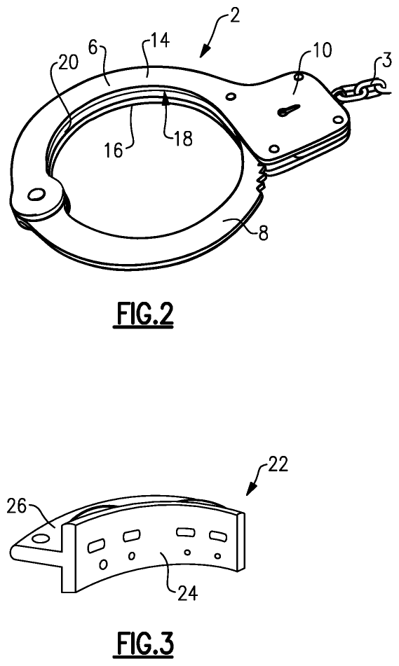

FIG. 2 is an illustration of one of the cuffs 2 with the two sections thereof 6 and 8 engages to form an area 12 within which a restrained individual's ankle or wrist may be engaged. As can be seen in FIG. 2 the first cuff section 6 has an upper member 14 and a lower member 16 disposed parallel to each other and which upper 14 and lower 16 members forms a slot 18 there between with slot 18 containing on one end thereof, the locking mechanism 10 which receives and locks into position the free end of the hinged section 8 and along a central portion thereof 20 a free open an unobstructed volume, which open volume 20 may receive the sensing module 22 of the present invention.

FIG. 3 depicts the sensing module 22 of the present invention. This module is comprised of a first sensor containing member 24 and a second flange member 26, perpendicular to the sensor containing member sized and shaped to frictionally be engaged into the open slot volume 20. In that way the sensor containing member 24 will positioned directly adjacent to the restrained individual's wrist or ankle when the handcuffs 1 containing said sensing module 22 are place on such individual.

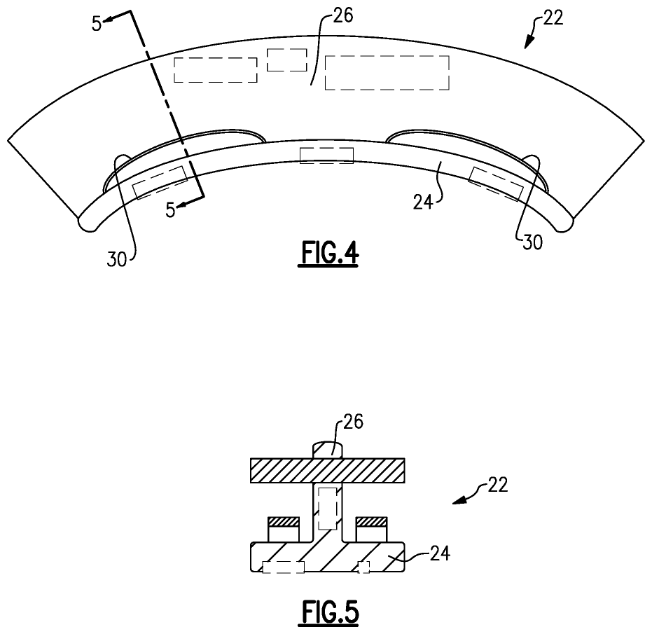

FIG. 4 is a side view and FIG. 5 is a cross-sectional view of the sensing module 22 of the present invention. The module may advantageously include clips 28 and springs 30 that will aid in maintaining said module 22 within slot volume 20 in addition to the module's friction fit within the slot volume 20.

The sensing module 22 of the present invention contains means for sensing data and conditions of interest about the restrained individual such as biometric data and location data as well as information about the status of the hand cuffs 1 themselves. The sensing means consist of one or more sensors installed withing the sensing module 22. The design and implementation of such sensors are well known in the art and may be of a variety of types including motion detectors (acceleration, vibration for example which can detect movement of the restrained individual as well as movement of hand cuffs 1 relative to the restrained individual), location sensors (GPS based for example) and biometric detecting sensors (heart rate, 02 level and the like). It will be clear to those skilled in the art that any attribute that may be sensed with some form of sensor may be incorporated into the present invention within the teaching of this invention. The sensing element may be powered in any way known in the art and most advantageously by an “on-board” rechargeable battery.

The sensing element of the subject device may also contain a pressure switch, of a type know in the art so that the sensing element is activated only when the restraining device of the present invention is actually in use. This, among other things, we “save” the battery life of the battery used to power the sensing element.

The sensing module 22 will also advantageously contain means, known in the art, for transmitting the sensed data from the module 22 to a receiver for receiving and processing the data. In actual operation, an individual restrained using the handcuffs 1 of the present invention containing the sensing module 22 can be very advantageously employed in keeping track of restrained individuals as to location and movement but may also be used to provide biometric data about such persons. While the sensing module 22 of the present invention may be manufactured as a component part of a restraining device, the present invention allows for the benefits of the present invention to be realizes using a standard issue set of hand cuffs 1 as are readily available on the market now without the need to alter or modify such handcuffs.

As described above, the device and system of the subject invention is most advantageously employed with a standard set of hand cuffs. However, any apparatus (such as a bracelet, wrist band, watch band or the like) that locates the device of the present invention in sufficient proximity to the monitored individual such that such individual may be monitored biometrically or as to location can be achieve, is within the scope of this invention.

Furthermore, it will be clear to one skilled in the art that the summary, detailed description, drawings, and claims provided herein make reference to particular features and methods of employing and deploying the subject apparatus. It is understood that the disclosure of the invention in this specification includes all possible combinations of such particular features. Moreover, many modifications and variations of the present invention are possible in light of the above teachings. Therefore, within the scope of the appended claims, the present invention may be practiced other than as specifically described.

What is claimed is:

1. A system for restraining an individual which provides information concerning a condition of interest about the restrained individual comprising:
a sensing element that senses the condition of interest;
a restraining device contain the sensing element and maintain such sensing element in proximity to the individual restrained so as to be able to sense such conditions of interest;
a communication component that communicates a sensed condition; and
a monitoring component that receives the sensed condition in a manner that can be monitored and displayed.

2. The system of claim 1, wherein the sensing element is removable installed within the restraining device.

3. The system of claim 1, wherein the restraining device is a standard set of handcuffs.

4. The system of claim 1, wherein the sensing element is designed to monitor biometric data from the restrained individual.

5. The system of claim 1, wherein the sensing element is designed to monitor location data from the restrained individual.
